SEQUENCE LISTING (11 sequences)

SEQ ID NO: 1 - protein, 339 aa, Homo sapiens
MSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 2 - protein, 339 aa, Artificial Sequence
  Description of Artificial Sequence Synthetic polypeptide
MSTVHEILCKLSLEGDHSTPPSAYGSVKAYTNFDAERDALNIETAIKTKGVDEVTIVNILTARSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 3 - protein, 329 aa, Artificial Sequence
  Description of Artificial Sequence Synthetic polypeptide
LSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 4 - protein, 329 aa, Artificial Sequence
  Description of Artificial Sequence Synthetic polypeptide
LSLEGDHSTPPSAYGSVKAYTNFDAERDALNIETAIKTKGVDEVTIVNILTARSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 5 - protein, 311 aa, Artificial Sequence
  Description of Artificial Sequence Synthetic polypeptide
AYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 6 - protein, 311 aa, Artificial Sequence
  Description of Artificial Sequence Synthetic polypeptide
AYTNFDAERDALNIETAIKTKGVDEVTIVNILTARSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 7 - DNA, 1035 bp, Artificial Sequence
  Description of Artificial Sequence Synthetic polynucleotide
atgtctactgttcacgagattttgtgtaagttgtctttggagggtgaccactctactccaccatctgcttacggttctgttaaggcttacactaacttcgacgctgagagagacgctttgaacattgagactgctattaagactaagggtgttgacgaggttactattgttaacattttgactaacagatctaacgctcaaagacaagacattgctttcgcttaccaaagaagaactaagaaggagttggcttctgctttgaagtctgctttgtctggtcacttggagactgttattttgggtttgttgaagactccagctcaatacgacgcttctgagttgaaggcttctatgaagggtttgggtactgacgaggactctttgattgagattatctgttctagaactaaccaagagttgcaagagattaacagagtttacaaggagatgtacaagactgacttggagaaggacattatctctgacacttctggtgacttcagaaagttgatggttgctttggctaagggtagaagagctgaggacggttctgttattgactacgagttgattgaccaagacgctagagacttgtacgacgctggtgttaagagaaagggtactgacgttccaaagtggatttctattatgactgagagatctgttccacacttgcaaaaggttttcgacagatacaagtcttactctccatacgacatgttggagtctattagaaaggaggttaagggtgacttggagaacgctttcttgaacttggttcaatgtattcaaaacaagccattgtacttcgctgacagattgtacgactctatgaagggtaagggtactagagacaaggttttgattagaattatggtttctagatctgaggttgacatgttgaagattagatctgagttcaagagaaagtacggtaagtctttgtactactacattcaacaagacactaagggtgactaccaaaaggctttgttgtacttgtgtggtggtgacgaccaccaccaccaccaccac

SEQ ID NO: 8 - DNA, 45 bp, Artificial Sequence
  Description of Artificial Sequence Synthetic primer
actattgttaacattttgactgctagatctaacgctcaaagacaa

SEQ ID NO: 9 - DNA, 45 bp, Artificial Sequence
  Description of Artificial Sequence Synthetic primer
ttgtctttgagcgttagatctagcagtcaaaatgttaacaatagt

SEQ ID NO: 10 - protein, 6 aa, Artificial Sequence
  Description of Artificial Sequence Synthetic His tag
  SITE (1)..(6): This sequence may encompass 2-6 residues
HHHHHH

SEQ ID NO: 11 - protein, 6 aa, Artificial Sequence
  Description of Artificial Sequence Synthetic 6xHis tag
HHHHHH

(19) United States
(12) Patent Application Publication
(10) Pub. No.: US 2023/0068460 A1
(43) Pub. Date: Mar. 2, 2023

(54) PROPHYLACTIC USES OF ANNEXIN A2

(71) Applicant: The Regents of the University of Colorado, a body corporate, Denver, CO (US)

(72) Inventors: Zhirui Wang, Denver, CO (US); Ernest Moore, Denver, CO (US)

(21) Appl. No.: 17/797,663

(22) PCT Filed: Feb. 4, 2021
(86) PCT No.: PCT/US21/16629

§ 371 Date: Aug. 4, 2022

Related U.S. Application Data
(60) Provisional application No. 62/970,058, filed on Feb. 4, 2020.

(51) Int. Cl. A61K 38/17; A61K 38/48; A61P 7/02

(52) U.S. Cl. CPC A61K 38/1709; A61K 38/482; A61P 7/02

(57) ABSTRACT

Curative and prophylactic therapies for microvascular and/or macrovascular thrombosis are provided, as are prophylactic therapies capable of preventing fibrinolysis shutdown and therapies for non-thrombotic disorders. Methods provide for administering a therapeutically effective amount of an annexin A2.

### SEQUENCE LISTING

The instant application contains a Sequence Listing which has been submitted via EFS-Web and is hereby incorporated by reference in its entirety. The ASCII copy, created on Feb. 4, 2021, is named 517709_34_SL, and is 20267 bytes in size.

### BACKGROUND

Prevention and treatment of microvascular or macrovascular thrombotic events due to the conditions of hypofibrinolysis and fibrinolysis shutdown using fibrinolytic agents is an active area of experimentation. However, advances have stagnated due to the high risk of bleeding complications with these agents and their poor efficacy in some individuals.

Annexin A2 (A2) is a highly conserved multifunctional protein located at the cell surface and intracellularly. Studies have shown that A2, with the help of S100 dimerization, serves as a platform at the cell surface for plasminogen and tissue plasminogen activator (tPA) colocalization. The heterodimer of S100-A2 normally produced via endothelial cells serves as a source of plasmin leading to fibrinolysis. However, there is limited data evaluating their individual coagulation properties.

### SUMMARY

In a first example (“Example 1”), provided herein is a method of preventing clot formation in a subject, including administering to the subject a therapeutically effective amount of an annexin A2.

In another example (“Example 2”), further to Example 1, the subject is selected as a candidate for thromboprophylaxis therapy.

In another example (“Example 3”), further to Example 1 or Example 2, the subject has fibrinolytic shutdown or is at risk of fibrinolytic shutdown.

In another example (“Example 4”), provided herein is a method of treating, preventing, or slowing progression of a non-thrombotic disorder of fibrin deposition in a subject in need thereof, including administering to the subject a therapeutically effective amount of an annexin A2.

In another example (“Example 5”), further to Example 4, the subject has acute respiratory distress syndrome, acute kidney injury, liver failure, or encapsulating peritoneal sclerosis.

In another example (“Example 6”), provided herein is a method of enhancing clot dissolution in a subject, including administering to the subject a therapeutically effective amount of annexin A2.

In another example (“Example 7”), further to Example 6, the subject is selected as a candidate for fibrinolytic therapy.

In another example (“Example 8”), further to Example 6 or Example 7, the subject has fibrinolytic shutdown.

In another example (“Example 9”), further to any one of Examples 1-8, the annexin A2 is a wild-type annexin A2 or a recombinant annexin A2.

In another example (“Example 10”), further to any one of Examples 1-9, the annexin A2 is a recombinant annexin A2 comprising a conservative substitution mutation at position N62, at position S64, or at both position N62 and S64, wherein the conservative substitution mutation is relative to SEQ ID NO: 1.

In another example (“Example 11”), further to any one of Examples 1-10, the the conservative substitution mutation at N62 is N62A or N62G, and the conservative substitution at S64 is S64A or S64G.

In another example (“Example 12”), further to any one of Examples 1-11, the annexin A2 is an annexin A2 comprising an amino acid sequence having at least 95% sequence identity to SEQ ID NO: 1, SEQ ID NO: 2, SEQ ID NO: 3, SEQ ID NO: 4, SEQ ID NO: 5, or SEQ ID NO: 6.

In another example (“Example 13”), further to any one of Examples 1-12, the effective amount of annexin A2 is between about 0.1 mg/kg to about 5 mg/kg.

In another example (“Example 14”), further to any one of Examples 1-13, the method further includes administering to the subject a therapeutically effective amount of one or more fibrinolytics

In another example (“Example 15”), further to Example 14, the one or more fibrinolytics are selected from the group consisting of: streptokinase, urokinase, anistreplase, alteplase, reteplase, and tenecteplase

### BRIEF DESCRIPTION OF THE DRAWINGS

The accompanying drawings are incorporated into and form a part of the specification to illustrate several examples of the present disclosure. The drawings simply illustrate examples of the disclosure and are not to be construed as limiting the disclosure to the illustrated and described examples. Further features and advantages will become apparent from the following, more detailed, description of the various aspects, embodiments, and configurations of the disclosure, as illustrated by the drawings referenced below.

FIGS. 1A-1C are photographs of an SDS-PAGE gel (FIG. 1A) and Western blots (FIGS. 1B and 2C) demonstrating the expression and purification of glycosylated (lanes 2 and 3 of each panel) and non-glycosylated (lanes 4 and 5 of each panel) annexin A2.

FIG. 2 depicts a series of representative thromboelastography tracings for each of a control (Cont) sample, a sample treated with tissue plasminogen activator (tPA), and a sample treated with tPA and annexin A2. Also depicted is a table providing the R time (minutes), angle (degrees), maximum amplitude (MA; millimeters), and the percentage of clot lysed after 30 minutes (Ly30; percentage) for the three traces.

FIGS. 3A-3D are bar graphs illustrating fibrinolysis (LY30) measured by thromboelastograph in whole blood samples treated with control (cont), vehicle (veh), tPA alone, or the noted concentration of non-glycosylated or glycosylated annexin A2, with or without tPA as indicated. FIG. 3A: non-glycosylated annexin A2 with tPA; FIG. 3B: non-glycosylated annexin A2 without tPA; FIG. 3C: glycosylated annexin A2 with tPA; FIG. 3D: glycosylated annexin A2 without tPA. ****=P<0.0001; relative to tPA alone.

FIGS. 4A-4D are bar graphs illustrating clotting time (R time) measured by thromboelastograph in whole blood samples treated with control (cont), vehicle (veh), tPA alone, or the noted concentration of non-glycosylated or glycosylated annexin A2, with or without tPA as indicated. FIG. 4A: non-glycosylated annexin A2 with tPA; FIG. 4B: non-glycosylated annexin A2 without tPA; FIG. 4C: glycosylated annexin A2 with tPA; FIG. 4D: glycosylated annexin A2 without tPA. *=P<0.05; **=P<0.01; ***=P<0.001; relative to tPA alone.

FIGS. 5A-5D are bar graphs illustrating maximum amplitude (MA) measured by thromboelastograph in whole blood samples treated with control (cont), vehicle (veh), tPA alone, or the noted concentration of non-glycosylated or glycosylated annexin A2, with or without tPA as indicated. FIG. 5A: non-glycosylated annexin A2 with tPA; FIG. 5B: non-glycosylated annexin A2 without tPA; FIG. 5C: glycosylated annexin A2 with tPA; FIG. 5D: glycosylated annexin A2 without tPA. *=P<0.05****=P<0.0001; relative to tPA alone.

FIGS. 6A-6D are bar graphs illustrating angle measured by thromboelastograph in whole blood samples treated with control (cont), vehicle (veh), tPA alone, or the noted concentration of non-glycosylated or glycosylated annexin A2, with or without tPA as indicated. FIG. 6A: non-glycosylated annexin A2 with tPA; FIG. 6B: non-glycosylated annexin A2 without tPA; FIG. 6C: glycosylated annexin A2 with tPA; FIG. 6D: glycosylated annexin A2 without tPA. * =P<0.05; ** =P<0.01; relative to tPA alone.

### DETAILED DESCRIPTION

In the following sections, various compositions and methods are described in order to detail various embodiments. Practicing the various embodiments does not require the employment of all of the specific details outlined herein, but rather concentrations, times, and other specific details may be modified. In some cases, well known methods or components have not been included in the description.

As used herein, “treat” in reference to a condition means: (1) to ameliorate or prevent the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms or effects associated with the condition, and/or (4) to slow the progression of the condition or one or more of the biological manifestations of the condition. The terms “prevent,” “preventing,” and the like are to be understood to refer to a method of blocking the onset of disease or condition and/or its attendant symptoms. “Prevent” also encompasses delaying or otherwise impeding the onset of a disease or condition and/or its attendant symptoms.

As used herein, “therapeutically effective amount” in reference to an agent means an amount of the agent sufficient to treat the subject's condition but low enough to avoid serious side effects at a reasonable benefit/risk ratio within the scope of sound medical judgment. The safe and effective amount of an agent will vary with the particular agent chosen (e.g. consider the potency, efficacy, and half-life of the compound); the route of administration chosen; the condition being treated; the severity of the condition being treated; the age, size, weight, and physical condition of the patient being treated; the medical history of the patient to be treated; the duration of the treatment; the nature of concurrent therapy; the desired therapeutic effect; and like factors, but can nevertheless be determined by the skilled artisan.

For any compound, agent, or composition, the therapeutically effective amount can be estimated initially either in cell culture assays or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

A “subject” means any individual having, having symptoms of, or at risk for one or more of: 1) microvascular and/or macrovascular thrombosis, including venous thromboembolism; 2) fibrinolysis shutdown; 3) a non-thrombotic disorder of fibrin deposition such as, but not limited to, acute respiratory distress syndrome, and encapsulating peritoneal sclerosis. A subject may be human or non-human, and may include, for example, animals or species used as “model systems” for research purposes, such as a porcine model. In certain embodiments, the subject is a human patient having or at risk of developing one or more of: 1) microvascular and/or macrovascular thrombosis, including venous thromboembolism; 2) fibrinolysis shutdown; 3) a non-thrombotic disorder of fibrin deposition such as, but not limited to, acute respiratory distress syndrome, and encapsulating peritoneal sclerosis.

As used herein, a “pharmaceutical composition” is a formulation containing a compound or agent (e.g., annexin A2) in a form suitable for administration to a subject. Compounds and agents disclosed herein each can be formulated individually or in any combination into one or more pharmaceutical compositions. Accordingly, one or more administration routes can be properly elected based on the dosage form of each pharmaceutical composition. Alternatively, a compound or agent disclosed herein and one or more other therapeutic agents described herein can be formulated as one pharmaceutical composition.

The term “about” encompasses variations of ±10%, ±5%, ±2%, ±1%, ±0.5%, or ±0.1% of the numerical value of the number which the term “about” modifies.

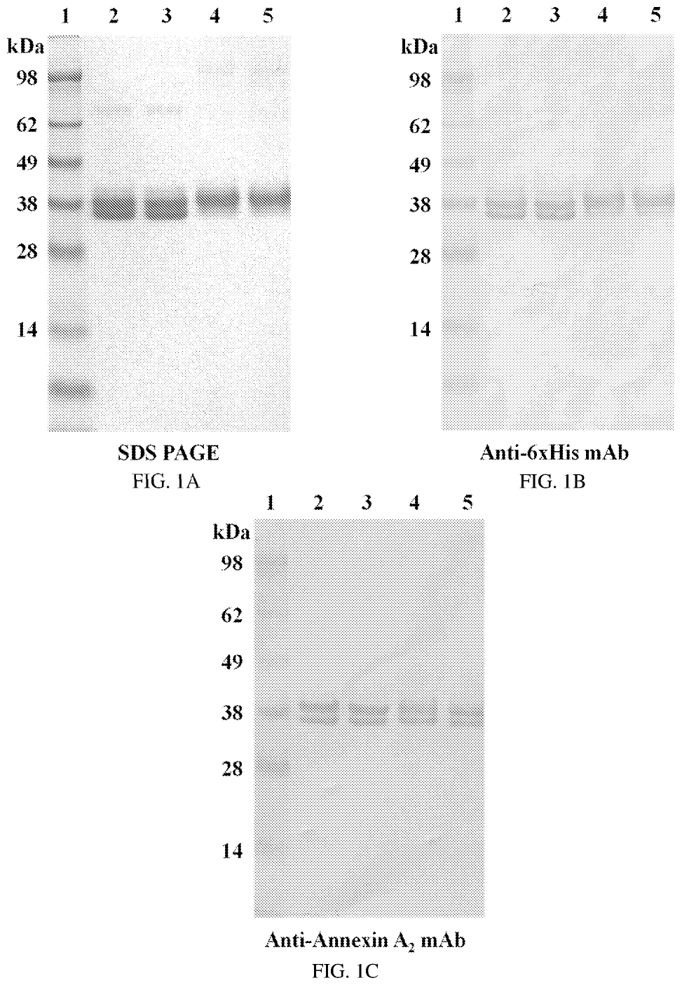

As illustrated by FIGS. 1-6 and described in Experimental Example 1, cell free annexin A2 significantly increases the fibrinolytic properties of tPA. Annexin A2′ s ability to enhance the activity of fibrinolytic agents, such as tPA, allows for far lower (i.e., safer) doses of fibrinolytic agents than are can be otherwise employed, thus mitigating bleeding risk. The use of annexin A2 as a pre- or co-treatment to increase efficacy of tPA in tPA-resistant clots (as sometimes seen in stroke patients, for example) without having to increase the treatment dose of tPA can avoid the risks associates with a high-dose tPA therapy. Pre- or co-treatment with annexin A2 can increase the efficacy of fibrinolytic agents in biochemical circumstances where they frequently perform poorly, such as in the perfusion of organs donated for transplant or in tissue grafts with small-caliber feeding vessels prone to states of low flow, where the application of leeches quite literally remains the state of the art. Use of annexin A2 can also increase safety of therapies in patients with impaired clot breakdown and also those with a high risk of bleeding due to underlying disease (e.g., kidney or liver disease and certain cancers).

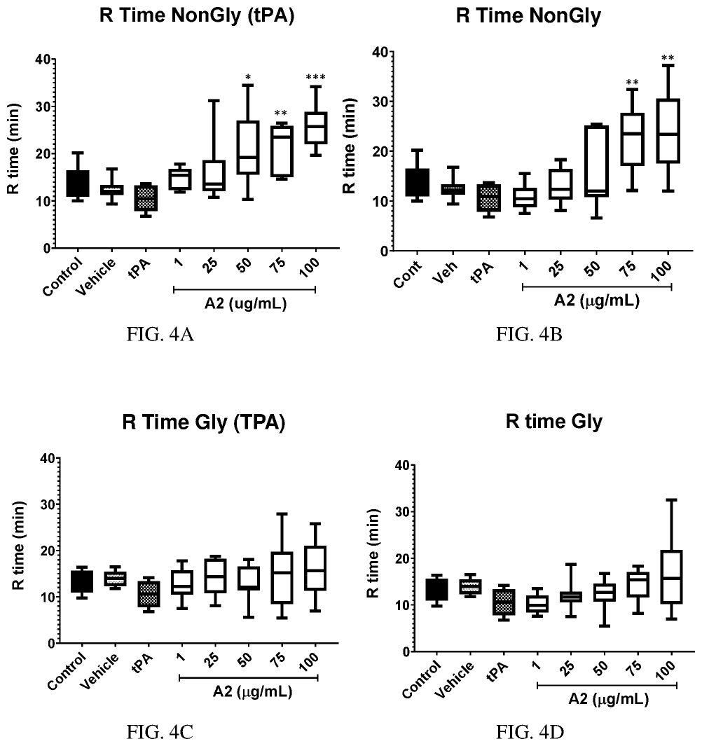

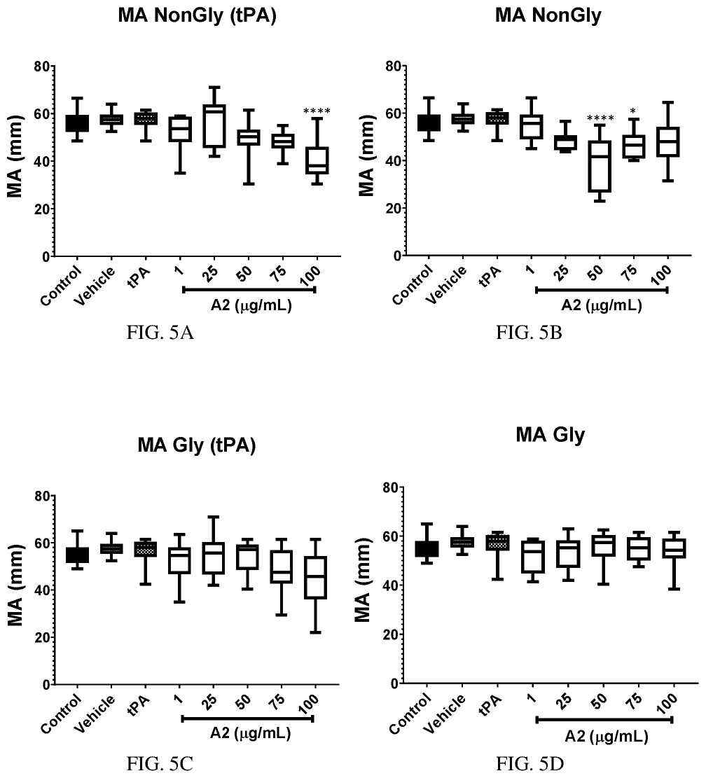

In evaluating annexin A2's effect on tPA, it was surprising found that annexin A2 can induce an anticoagulant effect regardless of the presence of tPA. This is evident in, for example, FIG. 4B, which shows annexin A2's ability to significantly increase the clotting time (R time), as measured by thromboelastography. Annexin A2 also decreases clot strength, as indicated by the decrease in maximum amplitude (FIG. 5B). While others have described the use of annexin A2 in treating existing thrombi, described herein for the first time is the use of annexin A2 monotherapy for the prophylactic prevention of blood clots, and its use in non-thrombotic disorders of fibrin deposition.

Embodiments of the present disclosure provide prophylactic therapies for microvascular and/or macrovascular thrombosis in a subject. Methods for preventing microvascular and/or macrovascular thrombosis is a subject are provided. The methods include administering to the subject a therapeutically effective amount of an annexin A2.

The methods described herein provide alternative methods of venous thromboembolism (VTE) prophylaxis (thromboprophylaxis). VTE is a principal cause of death in trauma patients, with trauma being a leading cause of death globally. The incidence of VTE without prophylaxis is as high as 80% after major trauma, and clinicians are often hesitant to begin timely VTE prophylaxis in critically injured patients. This is despite patients having a threefold greater risk of VTE if left without VTE prophylaxis for half of all days following admission to hospital. As described herein, annexin A2 monotherapy significantly delays and prevents clot formation, and reduces clot strength. Annexin A2 can thus be used to prevent or slow clotting in a subject.

A subject can be selected for treatment with annexin A2 in accordance with the present disclosure. In certain embodiments, the subject has suffered major trauma. The trauma can be blunt force trauma or penetrating trauma. The subject can be identified as a candidate for trauma-related thromboprophylaxis according to current practices. In other embodiments, the subject is to undergo a surgery during which VTE is an identified concern. Risk factors for venous thromboembolism include major medical illness, obesity, previous VTE, cancer, age over 60 years, prolonged immobilization, lower limb paralysis, use of hormonal therapy, and comorbid conditions such as stroke, congestive heart failure or recent myocardial infarction. Where it is desirable to prevent or slow clot formation in a subject, a therapeutically effective amount of annexin A2 is administered to the subject.

While trauma increases the risk of VTE, it can also cause trauma-induced coagulopathy (TIC), which encompasses a spectrum of coagulation changes after severe injury. This includes fibrinolysis shutdown, which is an acute pathological condition involving endogenous inhibition of the fibrinolytic system lasting for a few days to weeks following trauma, myocardial infarction, and elective surgery, for example. Thrombosis in the pulmonary vasculature occurs in nearly 25% of severely injured patients within 48 hours of injury, and microvascular clots in organs other than the lungs have been implicated in nonlung organ dysfunction. Thus, even in the setting of injury with hemorrhage, fibrinolysis is required for overall homeostasis to clear the microvasculature of excessive fibrin deposition. However, upwards of 65% of severely injured patients experience low fibrinolytic activity within 12 hours of injury. The resulting low rate of clot degradation is associated with increased mortality. In certain embodiments, acute fibrinolytic shutdown is defined as an LY30<0.5% in a rapid thromboelastogram. In other embodiments, acute fibrinolytic shutdown is defined as maximum lysis (ML) 1<5%, and a clot lysis index (CLI) >97%, as measured by rotational thromboelastography. Patients having or at risk of developing fibrinolytic shutdown can be treated by administering a therapeutically effective amount of annexin A2 to the patient. As provided by the examples, annexin A2 alone induces an anticoagulant effect. Without being bound by any particular theory, administration of annexin A2 may potentiate innate fibrinolytics such as tissue plasminogen activator (tPA).

Yet further embodiments of the present disclosure provide methods of treating a non-thrombotic disorder of fibrin deposition in a subject. The methods include administering to the subject a therapeutically effective amount of annexin A2. Examples of non-thrombotic disorders include, but are not limited to, acute respiratory distress syndrome, acute kidney injury, liver failure, and encapsulating peritoneal sclerosis.

Other embodiments provide methods for enhancing clot dissolution in a subject. The methods include administering to the subject a therapeutically effective amount of annexin A2. Enhancing clot dissolution can be beneficial for patients having microvascular and/or macrovascular thrombosis.

The annexin A2 to be administered to a subject in accordance with any of the methods described herein can be a wild-type annexin A2. That is, a form of annexin A2 that can be isolated from a biological sample. The wild-type annexin A2 can be a cell-free wild-type annexin A2. The wild-type annexin A2 to be administered to the subject can be an exogenous wild-type annexin A2.

The annexin A2 to be administered to a subject in accordance with a method described herein can be a recombinant annexin A2. Examples of recombinant annexin A2 are described in, for example, U.S. Pat. No. 9,314,500 and U.S. application Ser. No. 12/918,726, the contents of which are incorporated herein by reference in their entireties. The post-translational glycosylation patterns of the recombinant annexin A2 can be altered relative to wild-type annexin A2, as described in U.S. Pat. No. 9,314,500.

Annexin A2 is an endothelial cell surface receptor for both plasminogen and tPA, and facilitates the generation of plasmin. It is a calcium-dependent phospholipid-binding protein of about 339 amino acids in length. The wild-type annexin A2 gene has four variants: isoform 1 (NM_001002858.2; NP_001002858.1) is the longest isoform; isoform 2 (NM_001002857.1; NP_001002857.1) has an alternate 5′ UTR relative to isoform 1, and uses a downstream AUG start codon; isoform 3 (NM_004039.2; NP_004030.1) lacks a segment in the 5′ region as compared to variant 1 and uses the same downstream AUG start codon as isoform 2; isoform 4 (NM_001136015.2; NP_001129487.1) has an alternate 5′ UTR relative to isoform 1, and uses the same downstream AUG start codon as isoforms 2 and 3. The general amino acid sequence for wild-type annexin A2 is provided by SEQ ID NO:1.

In certain embodiments, one or both amino acids at positions 62 and 64 of SEQ ID NO: 1 are mutated, thereby disrupting the N-glycosylation site at those positions. The mutation at N62 can be to any amino acid other than N, so long as a desired activity of the resulting annexin A2 protein is retained or is enhanced Similarly, the mutation at S64 can be to any amino acid other than serine or threonine, so long as a desired activity of the resulting annexin A2 protein is retained or is enhanced. The substitution mutation can be a conservative mutation. The substation mutation at N62 can be, for example, to alanine, glycine, glutamine, aspartate, or glutamate. The substitution mutation at S64 can be, for example, to alanine, glycine, glutamine, aspartate, or glutamate.

The annexin A2 can be a full-length wild-type annexin A2 or recombinant annexin A2 having a substitution mutation at N62 and/or S64, or it can be a truncated version of wild-type annexin A2 or recombinant annexin A2 having a substitution mutation at N62 and/or S64. In certain embodiments, the annexin A2 is truncated immediately prior to L11. In other embodiments, the annexin A2 is truncated just prior to A29. A summary of representative forms of annexin A2 contemplated for use according to the methods described here is provided by Table 1.

TABLE 1
Forms of annexin A2 of use in the described methods.

| Annexin A2 | SEQ ID | |
|---|---|---|
| Description | NO: | Amino Acid Sequence |
| Wild-type, full-length | 1 | MSTVHEILCK LSLEGDHSTP PSAYGSVKAY TNFDAERDAL |
| | | NIETAIKTKG VDEVTIVNIL TNRSNAQRQD IAFAYQRRTK |
| | | KELASALKSA LSGHLETVIL GLLKTPAQYD ASELKASMKG |
| | | LGTDEDSLIE IICSRTNQEL QEINPVYKEM YKTDLEKDII |
| | | SDTSGDFRKL MVALAKGPPA EDGSVIDYEL IDQDARDLYD |
| | | AGVKRKGIDV PKWISIMTER SVPHLQKVFD RYKGYSPYDM |
| | | LESIRKEVKG DLENAPLNLV QCIQNKPLYF ADRLYDSMKG |
| | | KGTRDKVLIR IMVSPSEVDM LKIRSEFKRK YGKSLYYYIQ |
| | | QDTKGDYQKA LLYLCGGDD |
| N62A, Full-length | 2 | MSTVHEILCK LSLEGDHSTP PSAYGSVKAY TNFDAEPDAL |
| | | NIETAIKTKG VDEVTIVNIL TARSNAQRQD LAFAYQRRTK |
| | | KELASALKSA LSGHLETVIL GLLKTPQAYD ASELKASMKG |
| | | LGTDEDSLIE IICSPTNQEL QEINRVYKRM YKTDLEKDII |
| | | SDTSGDFPKL MVALALGRRA EDGSVIDYEL IDQDAPDLYD |
| | | AGVKRKGTDV PKWISIMTER SVPHLQKVFD RYKSYSPYDM |
| | | LESIPKEVKG KLENAFLMLV QCIQNKPLYF ADRLYDSMKG |
| | | KGTRDKVLIR IMVSRSEVDM LKIRSEFKRK YGKSLYYYIQ |
| | | QDTKGDYQKA LLYLCGGDD |
| Wild-type, L11 | 3 | LSLEGDHSTP PSAYGSVKAY TNFDAERDAL NIETAIKTKG |
| truncation | | VDEVTIVNIL INPSNAQRQD IAFAYQPPIK KELASALKSA |
| | | LSGHLETVIL GLLKTPAQYD ASELKASMKG LGTDEDSLIE |
| | | IICSRTNQEL QEINRVYKEM YKTDLEKDII SDTSGDFRKL |
| | | MVALAKGRRA EDGSVIDYEL YDQDARDLYD AGVKRKGTDV |
| | | PKWISIMTER SVPHLQKVFD RYKSYSPYDM LESIRKEVKG |
| | | DLENAFLNLV QCIQNKPLYF ADRLYDSMKG KGTRDKVLIR |
| | | IMVSRSEVDM LKIRSEFKRK YGKSLYYYIQ QDTKGDYQKA |
| | | LLYLCGGDD |
| N62A, L11 truncation | 4 | LSLEGDHSTP PSAYGSVKAY TNFDAERDAL NIETAIKTKG |
| | | VDEVTIVNIL TARSNAQRQD IAFAYQRRTK KELASALKSA |
| | | LSGRLETVIL GLLKTPAQYD ASELKASMKG LGTDEDSLIE |
| | | IICSRTNQEL QEINRVYKEM YKTDLEKDII SDTSGDFRKL |
| | | MVALAKGRRA EDGSVIDYEL IDQDARDLYD AGVKRKGTDV |
| | | PKWISIMTER SVPHLQKVFD PYKSYSPYDM LESIRKEVKG |
| | | DLENAFLNLV QCIQNKPLYF ADRLYDSMKG KGTRDKVLIR |
| | | IMVSRSEVDM LKIRSEFKRK YGKSLYYYIQ QDTKGDYQKS |
| | | LLYLCGGDD |
| Wild-type, A29 | 5 | AYTNFDAERD ALNIETAIKT KGVDEVTIVN ILTNRSNAQR |
| truncation | | QDIAFAYQRR TKKELASALK SALSGHLETV ILGLLKTPAQ |
| | | YDASELKASM KGLGTDEDSL TEIICSRTNQ ELQEINRVYK |
| | | EMYKTDLEKD IISDTSGDFP KLMVALAKGR RAEDGSVIDY |
| | | ELIDQDARDL YDAGVKRKGT DVPKWISIMT ERSVPHLQKV |
| | | FDRYKSYSPY DMLESIRKEV KGDLENAFLN LVQCIQNKPL |
| | | YFADRLYDSM KGKGTRDKVL IRIMVSPSEV DMLKIRSEFK |
| | | RKYGKSLYYY IQQDTKGDYQ KALLYLCGGD D |
| N62A, A29 truncation | 6 | AYTNFDAEPD ALNIETAIKT KGVDEVTIVN ILTARSNAQR |
| | | QDIAFAYQRR TKKELASALK SALSGHLETV ILGLLKTPAQ |
| | | YDASELKASM KGLGTDEDSL IEIICSRTNQ ELQEINPVYK |
| | | EMYKTDLEKD IISDTSGDFR KLMVALAKGP PAEDGSVIDY |
| | | ELIDQDARDL YDAGVKRKGI DVPKWISIMT ERSVPHLQKV |
| | | FDRYKSYSPY DMLESIRKEV KGDLENAFLN LVQCIQNKPL |
| | | YFADPLYDSM KGKGTRDKVL IRIMVSRSEV DMLKIRSEFK |
| | | RKYGKSLYYY IQQDTKGDYQ KALLYLCGGD D |

Generally, the annexin A2—whether wild-type or recombinant—is derived or otherwise developed from the same species to which the subject belongs. For example, if annexin A2 is to be administered to a human subject according to the methods described herein, the annexin A2 to be administered is a human annexin A2. However, in some embodiments, the annexin A2 to be administered to the subject is derived or otherwise developed from a species different than that of the subject. For example, a porcine annexin A2 can be administered to a human subject.

In some embodiments, the annexin A2 polypeptides described herein further include a polypeptide tag useful for purifying the annexin A2 protein. Examples of such purification polypeptide tags include polyhistidine tags (e.g., two, three, four, five, or six consecutive histidine residues (SEQ ID NO: 10) at the C-terminus), glutathione-S-transferase (GST), a FLAG tag, a haemagglutinin (HA) tag, or a myc tag. The annexin A2 can further include a proteolytic cleave site between the annexin A2 sequence and the purification tag, allowing for removal of the tag following purification.

The annexin A2 polypeptides of the disclosure can be expressed in any known expression system. To maximize expression in a given expression system, the nucleic acid sequence encoding the annexin A2 can be codon-optimized for the particular expression system to be used. In some embodiments, annexin A2 is expressed in a Pichia pastoris expression system. In such embodiments, the nucleic acid sequence encoding the annexin A2 is codon optimized for expression in P. pastoris. A nucleic acid sequence codon-optimized for expression of full-length, wild-type annexin A2 with a 6× His tag (SEQ ID NO: 11) in P. pastoris is provided by SEQ ID NO: 7.

Annexin A2 can be produced using protein production methods known to those of skill in the art. For example, a scaled-up fermentation expression method utilizing P. pastoris can be used. Vectors suitable for expressing annexin A2 in a selected expression system are known.

Annexin A2 can be purified according to methods known to those of skill in the art. For example, nickel-based purification methods can be used where the annexin A2 includes a polyhistidine tag. Other methods include ammonium sulfate precipitation, reversed phase chromatography, hydrophobic interaction chromatography, size exclusion chromatography, immunoaffinity chromatography, HPLC, or any of the purification tags described above.

The annexin A2 is administered to the subject in a therapeutically effective amount. In some embodiments, the annexin A2 is administered at a dose of about 0.1 mg/kg to about 5 mg/kg.

One or more additional compounds affecting fibrinolysis and/or fibrin deposition may be administered to the subject in addition to the annexin A2. In some embodiments, methods described herein further include administering to the subject a therapeutically effective amount of tissue plasminogen activator (tPA). Other compounds affecting fibrinolysis (i.e., adjuncts to fibrinolytic therapy) and/or fibrin deposition are also contemplated, such as streptokinase, urokinase, anistreplase, alteplase (a recombinant tPA), reteplase, and tenecteplase. Such compounds can be administered following administration of annexin A2, concurrently with administration of annexin A2, or prior to administration of annexin A2. In particular embodiments, the one or more additional compounds are administered following administration of annexin A2. The additional compounds can be administered at their standard, approved dosages, or at a lower dose. As described above and in the experimental examples, annexin A2 can affect the potency of at least tPA, allowing for lower doses to be used.

### EXAMPLES

The materials, methods, and embodiments described herein are further defined in the following Examples. Certain embodiments are defined in the Examples herein. It should be understood that these Examples, while indicating certain embodiments, are given by way of illustration only. From the disclosure herein and these Examples, one skilled in the art can ascertain the essential characteristics of this invention, and without departing from the spirit and scope thereof, can make various changes and modifications of the invention to adapt it to various usages and conditions.

### Experimental Example 1

### Cell Free Annexin A2 Enhances tPA Mediated Fibrinolysis Measured by Thromboelastography

### Methods

DNA Construction. Codon-optimized human annexin A2 DNA (339 amino acids, UniProtKB/Swiss-Prot P07355) was synthesized. To facilitate the downstream protein purification, six histidines (6× His tag (SEQ ID NO:11)) were added to the C-terminus. The synthesized human annexin A2 DNA carrying 6× His tag (SEQ ID NO:11) was sub-cloned into a yeast Pichia pastoris expression vector pwPlCZalpha between XhoI and EcoRI restriction sites. To construct the non-N-glycosylated human annexin A2, the unique asparagine (N-linked glycosylation site) was replaced with non-polarized alanine (N62A) using a site-directed mutagenesis kit. The site-directed mutagenesis primers were sense primer N62AFor (5′ ACT ATT GTT AAC ATT TTG ACT GCT AGA TCT AAC GCT CAA AGA CAA 3′; SEQ ID NO:8) and antisense primer N62ARev (5′ TTG TCT TTG AGC GTT AGA TCT AGC AGT CAA AAT GTT AAC AAT AGT 3′; SEQ ID NO: 9). The mutated human annexin A2 DNA construct was confirmed by sequencing.

Protein Expression. Glycosylated or non-N-glycosylated human annexin A2 DNA construct (˜5 μg) was linearized and transformed into yeast Pichia pastoris strain X33 using an electroporation system. Transformed yeast cells were spread on YPD agar plates (1% yeast extract, 2% peptone, 1.5% agar, 2% dextrose) containing 100 μg/mL of zeocin and incubated at 30° C. for 3-4 days. Six yeast colonies were randomly picked and cultured in tubes containing 5 mL of YPD (1% yeast extract, 2% peptone, 2% dextrose) at 30° C., 250 rpm for 24 h, then in YPG (1% yeast extract, 2% peptone, 1% glycerol) at 30° C., 250 rpm for another 24 h. The cultures were induced in 2 mL of BMMYC (1% yeast extract, 2% peptone, 100 mM potassium phosphate, pH 7.0, 1.34% yeast nitrogen base without amino acids, 4×10-5% biotin, 0.5% methanol and 1% casamino acids) for 48 h at 25° C., at 225 rpm. 0.5% methanol was added twice daily to sustain the methanol level. Antifoam was added in all the growth and induction media at 0.02%. 1 mM phenylmethanesulfonyl fluoride was added to inhibit the protein degradation for the induction phase. 100 units/mL of penicillin and 100 μg/mL of streptomycin were added to all the growth and induction media to suppress bacterial contamination. The culture supernatants were analyzed using 4-12% SDS gel under non-reducing conditions.

One clone was selected for large-scale expression. The large-scale expression was scaled-up from the small tube expression. An incubator shaker was used during incubation to express both glycosylated and non-N-glycosylated human annexin A2 on a large-scale. The seed culture was prepared by inoculating a single colony into YPD medium, then incubating at 25° C., 225 rpm overnight. 5% of the seed culture was transferred to 1L shake flasks containing 250 mL YPD medium and cultured at 30° C., 250 rpm, for 24 h. Subsequently, cells were centrifuged at 1500 rpm for 5 minutes and the cell pellet was re-suspended in 250 mL YPG medium and cultured at 30° C., 250 rpm, for 24 h. For induction phase, cells were centrifuged at 1500 rpm for 5 minutes and the cell pellet was re-suspended in 125 mL BMMYC induction medium and induced at 25° C., 225 rpm, for 48 hrs. During the induction phase, 0.5% methanol was added twice daily to sustain the methanol level. After induction, yeast cells were pelleted by centrifugation at 3000 rpm, 4° C., for 10 minutes. The supernatant, containing the human annexin A₂, was collected for the purification. Antifoam, PMSF and penicillin/streptomycin were added to the media at the same concentrations as for the small-scale expression.

Protein Purification. Ni-Sepharose™ 6 fast flow resin was packed in a 5 cm×20 cm XK50 column for the first step purification. The column was equilibrated with 20 mM Tris-HCl pH 7.4, 0.5 M NaCl, and 5 mM imidazole. The sample was prepared by adding final concentration of 20 mM Tris-HCl pH 7.4, 0.5 M NaCl and 5 mM imidazole, filtered through crepe fluted filter and loaded onto the equilibrated column. The column was washed using 20 mM Tris-HCl pH 7.4, 0.5 M NaCl, and 5 mM imidazole. The bound proteins were eluted with 20 mM Tris-HCl pH 7.4, 0.5 M NaCl, and 500 mM imidazole. The purification fractions were analyzed using 4-12% SDS gel. The fractions containing the protein of interest were pooled and dialyzed using a 3.5 kDa cut off Spectra/Por® membrane tubing (Spectrum Labs, Cincinnati, Ohio) against 20 mM Tris-HCl pH 8.0, 1 mM EDTA pH 8.0, 5% glycerol at 4° C. with constant stirring. The dialysis buffer was replaced once.

Strong cation exchange resin Poros® 50 HS (Applied Biosystems) in a XK16/20 column was used for the second step purification. The column was equilibrated with 20 mM Tris-HCl pH 8.0, 1 mM EDTA pH 8.0, and 5% glycerol. The dialyzed sample was loaded into the column and washed with 20 mM Tris-HClpH 8.0, 1 mM EDTA pH 8.0, and 5% glycerol. The bound protein was eluted with 50 then 100 mM sodium borate for glycosylated version or 100 then 200 mM sodium borate for non-N-glycosylated version in 20 mM Tris-HCl pH 8.0, 1 mM EDTA pH 8.0, and 5% glycerol. The purified fractions were analyzed using 4-12% SDS gel. For glycosylated version, we pooled all the elution fractions with 50 mM and 100 mM sodium borate as final product. For non-N-glycosylated version, we performed one more step purification. The pooled flow-through and washing fractions from the second step purification were collected and dialyzed using a 3.5 kDa cut off Spectra/Por® membrane tubing (Spectrum Labs, Cincinnati, Ohio) against 20 mM Tris-HCl pH 7.4, 0.5 M NaCl, 5 mM imidazole at 4° C. with constant stirring. The dialysis buffer was replaced once. The dialyzed sample was then further purified using Ni-Sepharose® 6 fast flow resin in a XK16/20 column as described for the first step purification. The pooled protein product was concentrated down with Centricon Plus-70 (10 kDa cut off, Millipore, Burlington, Mass.), dialyzed against PBS pH 7.4, and 5% glycerol, filter sterilized and stored at 80° C. freezer. Protein concentration was determined using the Pierce BCA protein assay kit (Thermo Fisher Scientific, Waltham, Mass.).

Western Blotting. Human annexin A2 protein samples were separated by electrophoresis using 4-12% SDS gel and the gel was then electro-transferred onto a nitrocellulose membrane filter paper. The membrane was then blocked using 5% non-fat dry milk in 1×PBS, 0.02% Tween 20 for 1 h with shaking and then washed once with 1×PBS, pH7.4, 0.02% Tween 20 at room temperature with shaking. Human annexin A2 were detected using mouse anti-His monoclonal antibody (Thermo Fisher Scientific) or anti-human annexin A2 mAb (Clone# 666316, R&D) as primary antibodies and rat anti-mouse IgG-HRP (Thermo Fisher Scientific) as secondary antibody in 5% non-fat dry milk in 1×PBS, 0.02% Tween 20. The protein bands were visualized by adding TMB membrane peroxidase substrate and the color development was stopped with dH₂O.

In vitro Thromboelastography. Venous blood samples from healthy volunteers were obtained from the antecubital fossa of the arm using a 21 G winged needle and collected in a blue top BD (Becton Dickinson, Franklin Lakes, N.H., USA) vacutainer containing 3.2% buffered sodium citrate solution (n=10).

Citrated native thromboelastography (TEG) assays were recalcified with 20 μL of 0.2M calcium chloride solution. Prior to the addition of blood, human recombinant tissue plasminogen activator (alteplase; Genentech, South San Francisco, Calif., USA) was prepared fresh in a glass vial and added to the TEG cup at a concentration of 150 ng/mL. Recombinant annexin A2 was added to the TEG cup containing calcium chloride and alteplase at increasing concentrations ranging from 1 μg/mL-100 μg/mL annexin A2 25 μg/mL increments and allowed to incubate for 5 minutes. Samples were run within 2 hours of collection. A total of 340 μL of volunteer blood was added to the TEG cup and ran per the manufacturer's instructions on a TEG 5000 Thromboelastograph Hemostasis Analyzer (Haemonetics, Niles, Ill.). The vehicle for A2 included 5% glycerol and phosphate buffer saline at physiologic pH 7.4. Vehicle alone was incubated in whole blood at a volume corresponding to the largest dose (100 μg/mL) of A2. Fibrinolysis was evaluated based on the percentage of clot lysis at 30 minutes after the clot achieved maximum strength (Ly30). The following thromboelastography measurements were recorded: clot initiation (R time), fibrin polymerization (angle) and clot strength (maximum amplitude (MA) clot lysis time 30 minutes after reaching MA (LY30)).

Statistical Analysis. Data were expressed as mean +s.e.m. ANOVA analysis followed by Tukey tests. Differences with P<0.05 were considered statistically significant.

### Results

Expression and Purification of Human Annexin A2. Codon-optimized glycosylated (i.e., wild-type) human annexin A2 DNA carrying a 6× His tag (SEQ ID NO: 11) at the C-terminus was synthesized and cloned into the yeast Pichia pastoris expression vector pwPlCZalpha. The 6× His tag (SEQ ID NO:11) was added at the C-terminus to facilitate protein purification. The non-N-glycosylated human annexin A2 was constructed to alleviate concerns that the N-linked high-mannose glycosylation using the yeast expression system might affect the function of the human annexin A2. The plasmid construction for the non-N-glycosylated human annexin A2 was the same as that for the glycosylated version, except for that the unique N-glycosylation site was replaced with non-polar amino acid alanine (N62A). Both glycosylated and non-N-glycosylated human annexin A2 were expressed using an established large-scale shake-flask yeast Pichia pastoris expression system. Ni-Sepharose 6 fast flow resin was used for the first-step purification and a strong cation-exchange resin Poros 50HS was used for the second-step purification. SDS-PAGE and Western blot analysis confirmed the expressed and purified both glycosylated and non-N-glycosylated human annexin A2 (FIGS. 1A-1C). Three bands were observed with the glycosylated version (lanes 2 and 3 of FIGS. 1A-1C), as previously described. Two bands were observed with the non-N-glycosylated version (lanes 4 and 5 of FIGS. 1A-1C). The N-linked glycosylation was also confirmed using Con-A Sepharose® 4B purification analysis. As previously reported, human annexin A2 expressed by Pichia pastoris was N-terminal truncated before Leu¹¹ or Ala²⁹. There are both N-linked glycosylated and non-N-glycosylated versions for each truncated format, with four bands total. However, the lower band of the glycosylated version is almost same size as the upper band of the non-N-glycosylated version (˜38 kDa). Therefore, only three bands were observed with the glycosylated version (˜40 kDa, ˜38 kDa and ˜36 kDa) (FIGS. 1A-1C). N-linked glycosylation analysis using Endo H and PNGase F confirmed the glycosylation state. Two non-N-glycosylated bands (˜38 kDa and ˜36 kDa) were observed after the treatment with Endo H and PNGase F, consistent with the previously reported two N-terminal-truncated non-N-glycosylated human annexin A2. The final product yield was ˜50 mg per liter of the harvested supernatant for the glycosylated version and —40 mg per liter for the non-N-glycosylated version.

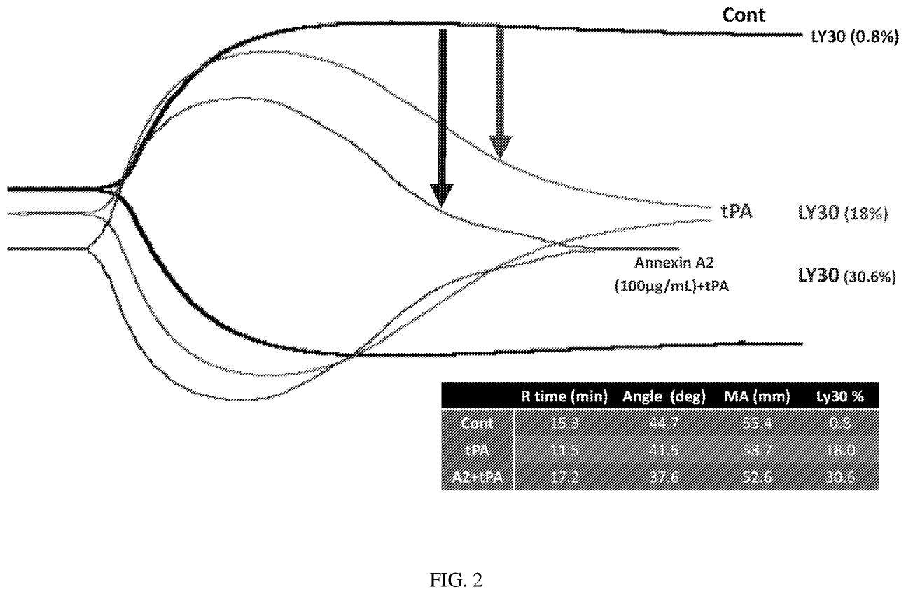

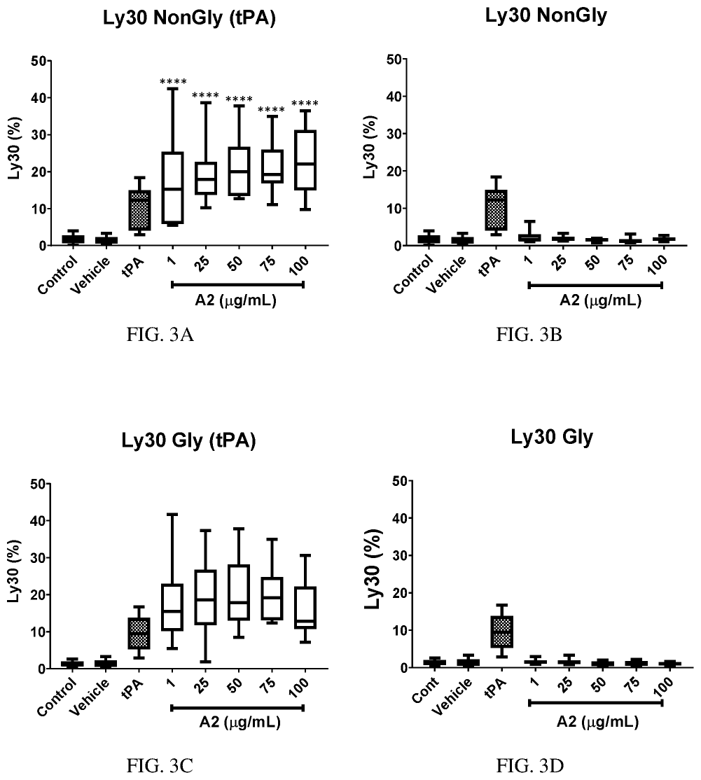

Thromboelastography Results. As depicted in FIG. 2, whole blood TEG used as a control (Cont) graphically demonstrated the typical champagne flute appearance with all parameters within normal limits When tPA was added to the whole blood, the appearance was that of a primary fibrinolysis. As depicted by FIGS. 2 and 3A-3D, tPA with whole blood caused a LY30% of 18%. When annexin A2 was added in the presence of tPA, the observed lysis was more pronounced with a lysis of 30.6%. vs 18% with tPA alone.

FIGS. 4A-4D depict clotting time (R time) with either N-linked glycosylated or non-N-glycosylated human annexin A2, with or without tPA as indicated. R value is time (in min) from the beginning of the trace until amplitude of 2 mm is reached. The R time with the non-glycosylated annexin A2 and tPA was significantly increased at concentrations of 50 μg/ml (p<0.05), 75 μg/ml (p<0.01), and 100 μg/ml (p<0.001) annexin A2. The R time with non-glycosylated annexin A2 without tPA showed a significant increase from baseline at 75 μg/ml and 100 μg/ml (p<0.01) annexin A2. The R time with the glycosylated form of A2 trended towards an elevation of R time at higher concentrations but never reached a level of significance. The addition of tPA had little to no effect on R time with or without the addition of glycosylated A2.

FIGS. 5A-5D depict clot strength, represented as maximum amplitude (MA), with either N-linked glycosylated or non-N-glycosylated human annexin A2, with or without tPA as indicated. MA is equal to the maximal width of the thromboelastograph and represents clot strength, with MA being proportional to clot strength. The MA with the non-glycosylated annexin A2 and tPA was significantly increased at a concentration of 100 μg/ml (p<0.001) annexin A2. The MA with non-glycosylated annexin A2 without tPA was significantly increased at concentrations of 50 μg/ml (p<0.001) and 75 μg/ml (p<0.01) annexin A2. the MA with the glycosylated form of A2 trended towards a decrease in MA at higher concentrations in combination with tPA, but never reached a level of significance. No effect was observed relative to baseline in the absence of tPA.

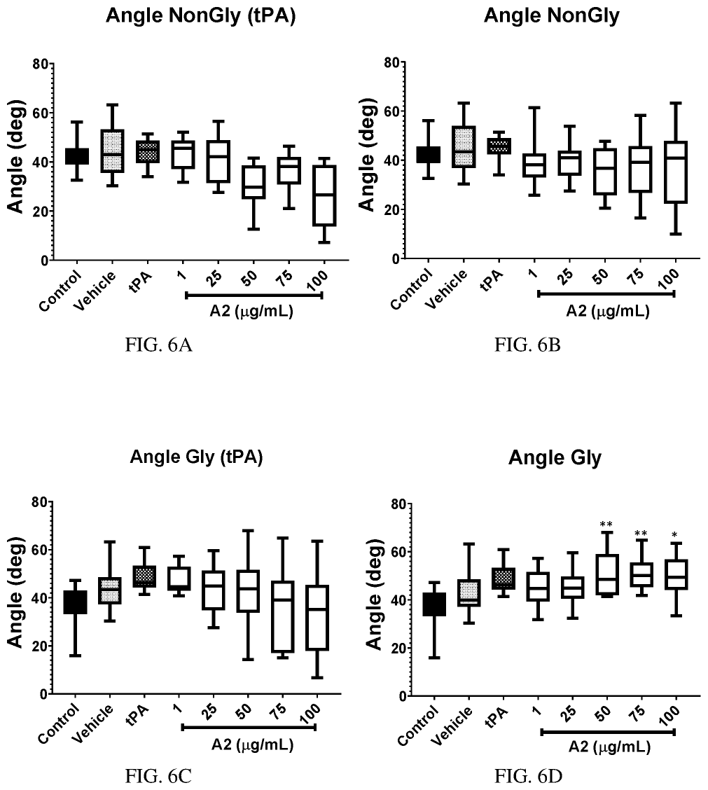

FIGS. 6A-6D depicts the angle with either N-linked glycosylated or non-N-glycosylated human annexin A2, with or without tPA as indicated. The angle correlates with the speed at which fibrin builds up and cross-linking occurs, and provides an indication of the rate of clot formation. The angle is tangent of the curve made as K is reached, where the K value is the time from the end of R until the clot reaches 20 mm. The angle with the non-glycosylated annexin A2 with tPA trended towards a decrease in angle at higher concentration of annexin A2 with non-glycosylated annexin A2, with and without tPA, but never reached a level of significance. A similar trend was seen with glycosylated annexin A2 and tPA. The angle observed with glycosylated annexin A2 in the absence of tPA was significantly increased at higher concentrations (50 μg/ml (p<0.01); 75 μg/ml (p<0.01); and 100 μg/ml (p<0.05) annexin A2).

### Discussion

Cell free non-glycosylated annexin A2 significantly increased the fibrinolytic properties of tPA, indicating that annexin A2 can be useful as a therapeutic to augment the lytic activity of endogenous tPA and reduce the use of exogenous tPA, thereby limiting the risk of bleeding. Surprisingly, it was also found that recombinant annexin A2 induced an anticoagulant effect in the absence of tPA.

1. A method of dissolving clot formation in a subject, comprising administering to the subject a therapeutically effective amount of an annexin A2.

2. The method of claim 1, wherein in the subject is selected as a candidate for thromboprophylaxis therapy.

3. The method of claim 1, wherein the subject has fibrinolytic shutdown or is at risk of fibrinolytic shutdown.

4. The method of claim 1, wherein the annexin A2 is a wild-type annexin A2 or a recombinant annexin A2.

5. The method of claim 1, wherein the annexin A2 is a recombinant annexin A2 comprising a conservative substitution mutation at position N62, at position S64, or at both position N62 and S64, wherein the conservative substitution mutation is relative to SEQ ID NO: 1.

6. The method of claim 5, wherein the conservative substitution mutation at N62 is N62A or N62G, and the conservative substitution at S64 is S64A or S64G.

7. The method of claim 1, wherein the annexin A2 is an annexin A2 comprising an amino acid sequence having at least 95% sequence identity to SEQ ID NO: 1, SEQ ID NO: 2, SEQ ID NO: 3, SEQ ID NO: 4, SEQ ID NO: 5, or SEQ ID NO: 6.

8. The method of claim 1, wherein the effective amount of annexin A2 is between about 0.1 mg/kg to about 5 mg/kg.

9. The method of claim 1, further comprising administering to the subject a therapeutically effective amount of one or more fibrinolytics.

10. The method of claim 9, wherein the one or more fibrinolytics are selected from the group consisting of: streptokinase, urokinase, anistreplase, alteplase, reteplase, and tenecteplase.

11. A method of treating, preventing, or slowing progression of a non-thrombotic disorder of fibrin deposition in a subject in need thereof comprising administering to the subject a therapeutically effective amount of an annexin A2.

12. The method of claim 11, wherein the subject has acute respiratory distress syndrome, acute kidney injury, liver failure, or encapsulating peritoneal sclerosis.

13. The method of claim 11, wherein the annexin A2 is a wild-type annexin A2 or a recombinant annexin A2.

14. The method of claim 11, wherein the annexin A2 is a recombinant annexin A2 comprising a conservative substitution mutation at position N62, at position S64, or at both position N62 and S64, wherein the conservative substitution mutation is relative to SEQ ID NO: 1.

15. The method of claim 14, wherein the conservative substitution mutation at N62 is N62A or N62G, and the conservative substitution at S64 is S64A or S64G.

16. The method of claim 11, wherein the annexin A2 is an annexin A2 comprising an amino acid sequence having at least 95% sequence identity to SEQ ID NO: 1, SEQ ID NO: 2, SEQ ID NO: 3, SEQ ID NO: 4, SEQ ID NO: 5, or SEQ ID NO: 6.

17. The method of claim 11, wherein the effective amount of annexin A2 is between about 0.1 mg/kg to about 5 mg/kg.

18. The method of claim 11, further comprising administering to the subject a therapeutically effective amount of one or more fibrinolytics.

19. The method of claim 18, wherein the one or more fibrinolytics are selected from the group consisting of: streptokinase, urokinase, anistreplase, alteplase, reteplase, and tenecteplase.

20. A method of enhancing clot dissolution in a subject, comprising administering to the subject a therapeutically effective amount of an annexin A2.

21. The method of claim 20, wherein the subject is selected as a candidate for fibrinolytic therapy.

22. The method of claim 20, wherein the subject has fibrinolytic shutdown.

23. The method of claim 20, wherein the annexin A2 is a wild-type annexin A2 or a recombinant annexin A2.

24. The method of claim 20, wherein the annexin A2 is a recombinant annexin A2 comprising a conservative substitution mutation at position N62, at position S64, or at both position N62 and S64, wherein the conservative substitution mutation is relative to SEQ ID NO: 1.

25. The method of claim 24, wherein the conservative substitution mutation at N62 is N62A or N62G, and the conservative substitution at S64 is S64A or S64G.

26. The method of claim 20, wherein the annexin A2 is an annexin A2 comprising an amino acid sequence having at least 95% sequence identity to SEQ ID NO: 1, SEQ ID NO: 2, SEQ ID NO:
3, SEQ ID NO: 4, SEQ ID NO: 5, or SEQ ID NO: 6.

27. The method of claim 20, wherein the effective amount of annexin A2 is between about 0.1 mg/kg to about 5 mg/kg.

28. The method of claim 20, further comprising administering to the subject a therapeutically effective amount of one or more fibrinolytics.

29. The method of claim 28, wherein the one or more fibrinolytics are selected from the group consisting of: streptokinase, urokinase, anistreplase, alteplase, reteplase, and tenecteplase.
